(19) United States
(12) Patent Application Publication
(10) Pub. No.: US 2009/0281322 A1
(43) Pub. Date: Nov. 12, 2009

(54) THERAPEUTICALLY USEFUL SUBSTITUTED 1,7-DIPHENYL-1,2,3,5,6,7-HEXAHYDROPYRIDO[3,2,1-Ij]QUINOLINE COMPOUNDS

(75) Inventors: Santosh C. Sinha, Ladera Ranch, CA (US); Smita S. Bhat, Irvine, CA (US); Ken Chow, Newport Coast, CA (US); Richard L. Beard, Newport Beach, CA (US); John E. Donello, Dana Point, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/433,978

(22) Filed: May 1, 2009

Related U.S. Application Data
(60) Provisional application No. 61/051,533, filed on May 8, 2008.

(51) Int. Cl. C07D 471/06

(52) U.S. Cl. 546/94

(57) ABSTRACT

Disclosed herein are compounds represented by the structural formula:

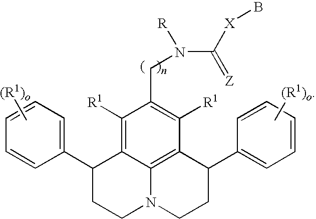

Therapeutic methods, compositions, and medicaments related thereto are also disclosed.

### RELATED APPLICATION

This application claims the benefit of U.S. Provisional Application Ser. No. 61/051,533, filed May 8, 2008, the disclosure of which is hereby incorporated in its entirety herein by reference

### BACKGROUND

Sphingosine is a compound having the chemical structure shown in the general formula described below, in which Y¹ is hydrogen. It is known that various sphingolipids, having sphingosine as a constituent, are widely distributed in the living body including on the surface of cell membranes of cells in the nervous system.

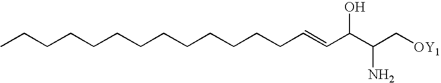

A sphingolipid is one of the lipids having important roles in the living body. A disease called lipidosis is caused by accumulation of a specified sphingolipid in the body. Sphingolipids present on cell membranes function to regulate cell growth; participate in the development and differentiation of cells; function in nerves; are involved in the infection and malignancy of cells; etc. Many of the physiological roles of sphingolipids remain to be solved. Recently the possibility that ceramide, a derivative of sphingosine, has an important role in the mechanism of cell signal transduction has been indicated, and studies about its effect on apoptosis and cell cycle have been reported.

Sphingosine-1-phosphate is an important cellular metabolite, derived from ceramide that is synthesized de novo or as part of the sphingomeyeline cycle (in animals cells). It has also been found in insects, yeasts and plants.

The enzyme, ceramidase, acts upon ceramides to release sphingosine, which is phosphorylated by sphingosine kinase, a ubiquitous enzyme in the cytosol and endoplasmic reticulum, to form sphingosine-1-phosphate. The reverse reaction can occur also by the action of sphingosine phosphatases, and the enzymes act in concert to control the cellular concentrations of the metabolite, which concentrations are always low. In plasma, such concentration can reach 0.2 to 0.9 μM, and the metabolite is found in association with the lipoproteins, especially the HDL. It should also be noted that sphingosine-1-phosphate formation is an essential step in the catabolism of sphingoid bases.

Like its precursors, sphingosine-1-phosphate is a potent messenger molecule that perhaps uniquely operates both intra- and inter-cellularly, but with very different functions from ceramides and sphingosine. The balance between these various sphingolipid metabolites may be important for health. For example, within the cell, sphingosine-1-phosphate promotes cellular division (mitosis) as opposed to cell death (apoptosis), which it inhibits. Intracellularly, it also functions to regulate calcium mobilization and cell growth in response to a variety of extracellular stimuli. Current opinion appears to suggest that the balance between sphingosine-1-phosphate and ceramide and/or sphingosine levels in cells is critical for their viability. In common with the lysophospholipids, especially lysophosphatidic acid, with which it has some structural similarities, sphingosine-1-phosphate exerts many of its extra-cellular effects through interaction with five specific G protein-coupled receptors on cell surfaces. These are important for the growth of new blood vessels, vascular maturation, cardiac development and immunity, and for directed cell movement.

Sphingosine-1 phosphate is stored in relatively high concentrations in human platelets, which lack the enzymes responsible for its catabolism, and it is released into the blood stream upon activation of physiological stimuli, such as growth factors, cytokines, and receptor agonists and antigens. It may also have a critical role in platelet aggregation and thrombosis and could aggravate cardiovascular disease. On the other hand the relatively high concentration of the metabolite in high-density lipoproteins (HDL) may have beneficial implications for atherogenesis. For example, there are recent suggestions that sphingosine-1-phosphate, together with other lysolipids such as sphingosylphosphorylcholine and lysosulfatide, are responsible for the beneficial clinical effects of HDL by stimulating the production of the potent antiatherogenic signaling molecule nitric oxide by the vascular endothelium. In addition, like lysophosphatidic acid, it is a marker for certain types of cancer, and there is evidence that its role in cell division or proliferation may have an influence on the development of cancers. These are currently topics that are attracting great interest amongst medical researchers, and the potential for therapeutic intervention in sphingosine-1-phosphate metabolism is under active investigation.

Fungi and plants have sphingolipids and the major sphingosine contained in these organisms has the formula described below. It is known that these lipids have important roles in the cell growth of fungi and plants, but details of the roles remain to be solved.

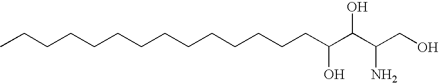

Recently it has been known that derivatives of sphingolipids and their related compounds exhibit a variety of biological activities through inhibition or stimulation of the metabolism pathways. These compounds include inhibitors of protein kinase C, inducers of apoptosis, immuno-suppressive compounds, antifungal compounds, and the like. Substances having these biological activities are expected to be useful compounds for various diseases.

### DESCRIPTION OF THE INVENTION

Disclosed herein are compound represented by the structural formula:

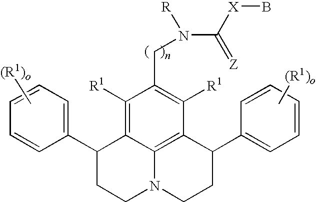

wherein n and o are independently 0, 1, 2, or 3;
R¹ is independently H or a substituent having a formula C₀₋₁₂H₀₋₃₀N₀₋₃O₀₋₅P₀₋₂S₀₋₃F₀₋₆Cl₀₋₃Br₀₋₃I₀₋₃;

### Z is O or S;

### X is O, S, or NR²;

R and R² are independently hydrogen or C₁₋₆ hydrocarbyl; and
B has a formula C₀₋₁₂H₁₋₃₀N₀₋₃O₀₋₅P₀₋₂S₀₋₃F₀₋₆Cl₀₋₃Br₀₋₃I₀₋₃, and is hydrogen, hydrocarbyl, heterohydrocarbyl, substituted or unsubstituted aryl, substituted or unsubstituted heteroaryl, or a combination thereof, wherein if X is NR², B and R² may together form a ring or ring system.

These compounds are useful for the treatment of diseases or conditions including: ocular disease, such as dry eye, glaucoma, neurodegenerative disease of the retina and/or optic nerve such as macular degeneration, including age-related macular degeneration, diabetic retinopathy, etc.; inflammation, including sepsis; angiogenesis; cardiovascular conditions and diseases; wounds; and pain. The compound is incorporated into a dosage form or a medicament and administered to the mammal, such as a person, in need thereof. Different types of suitable dosage forms and medicaments are well known in the art, and can be readily adapted for delivery of the compounds disclosed herein.

For the purposes of this disclosure, “treat,” “treating,” or “treatment” refer to the diagnosis, cure, mitigation, treatment, or prevention of disease or other undesirable condition.

Unless otherwise indicated, reference to a compound should be construed broadly to include compounds, pharmaceutically acceptable salts, prodrugs, tautomers, alternate solid forms, non-covalent complexes, and combinations thereof, of a chemical entity of a depicted structure or chemical name.

A pharmaceutically acceptable salt is any salt of the parent compound that is suitable for administration to an animal or human. A pharmaceutically acceptable salt also refers to any salt which may form in vivo as a result of administration of an acid, another salt, or a prodrug which is converted into an acid or salt. A salt comprises one or more ionic forms of the compound, such as a conjugate acid or base, associated with one or more corresponding counter-ions. Salts can form from or incorporate one or more deprotonated acidic groups (e.g. carboxylic acids), one or more protonated basic groups (e.g. amines), or both (e.g. zwitterions).

A prodrug is a compound which is converted to a therapeutically active compound after administration. For example, conversion may occur by hydrolysis of an ester group or some other biologically labile group. Prodrug preparation is well known in the art. For example, “Prodrugs and Drug Delivery Systems,” which is a chapter in Richard B. Silverman, Organic Chemistry of Drug Design and Drug Action, 2d Ed., Elsevier Academic Press: Amsterdam, 2004, pp. 496-557, provides further detail on the subject.

Tautomers are isomers that are in rapid equilibrium with one another. For example, tautomers may be related by transfer of a proton, hydrogen atom, or hydride ion.

Unless stereochemistry is explicitly and unambiguously depicted, a structure is intended to include every possible stereoisomer, both pure or in any possible mixture.

Alternate solid forms are different solid forms than those that may result from practicing the procedures described herein. For example, alternate solid forms may be polymorphs, different kinds of amorphous solid forms, glasses, and the like.

Non-covalent complexes are complexes that may form between the compound and one or more additional chemical species that do not involve a covalent bonding interaction between the compound and the additional chemical species. They may or may not have a specific ratio between the compound and the additional chemical species. Examples might include solvates, hydrates, charge transfer complexes, and the like.

In these compounds, n and o are independently 0, 1, 2, or 3. Thus, compounds represented by the structural formulas below are contemplated.

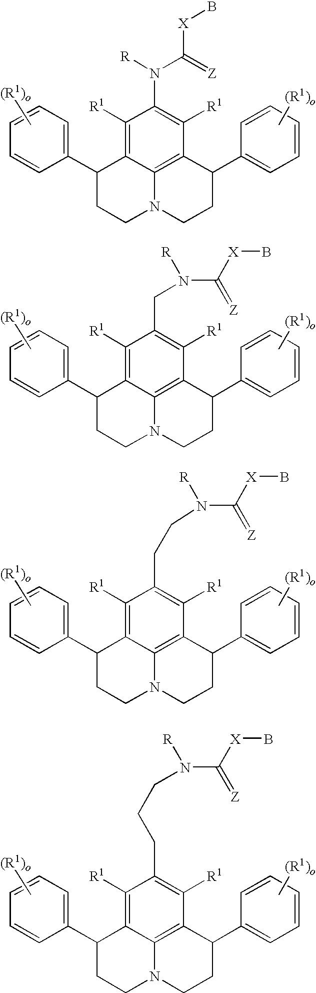

R¹ is independently H or a substituent having a formula C₀₋₁₂H₀₋₃₀N₀₋₃O₀₋₅P₀₋₂S₀₋₃F₀₋₆Cₗ₀₋₃Br₀₋₃I₀₋₃.

A substituent is a moiety attached to one or more ring carbons, and 2 or more substituents may themselves form an additional ring or rings incorporating the aryl or heteroaryl ring or ring system.

A substituent consists of: hydrogen, one or more hydrocarbyl fragments, one or more halogen atoms, and one or more functional groups, or combinations thereof.

Hydrocarbyl consists of carbon and hydrogen, wherein each carbon has 4 covalent bonds and each hydrogen has a single bond to a carbon atom. A double bond counts as 2 covalent bonds, and a triple bond counts as 3 covalent bonds. “Hydrocarbyl fragments” has the same meaning as “hydrocarbyl,” but is merely used for convenience for counting purposes. For example, one or more hydrocarbyl fragments means, 1, 2, or more distinct parts that each consist of hydrocarbyl, which may be interrupted by another moiety. For example, a functional group may be attached to 2 distinct hydrocarbyl fragments.

Hydrocarbyl includes, alkyl, alkenyl, alkynyl, aryl containing only hydrogen and carbon, and combinations thereof. Hydrocarbyl may be linear, branched, cyclic, or combinations thereof.

Alkyl is hydrocarbyl having no double bonds. Examples include methyl, ethyl, propyl isomers, butyl isomers, pentyl isomers, hexyl isomers, cyclopropyl, cyclobutyl, cyclopentyl, cyclohexyl, etc.

Alkenyl is hydrocarbyl having 1 or more double bonds. Examples include ethenyl, propenyl, butenyl isomers, pentenyl isomers, hexenyl isomers, cyclopentenyl, cyclohexenyl, etc.

Alkynyl is hydrocarbyl having 1 or more triple bonds. Examples include ethynyl, propynyl, butynyl isomers, pentynyl isomers, hexynyl isomers, cyclopentynyl, cyclohexynyl, etc.

Aryl is a substituted or unsubstituted aromatic ring or ring system. Although aryl may have any type of substituent defined herein, if hydrocarbyl includes aryl, the aryl will be unsubstituted or have hydrocarbyl fragments for substituents. Examples of aryl include substituted and unsubstituted phenyl, naphthyl, and biphenyl.

Each hydrogen atom has one covalent bond to carbon, nitrogen, oxygen, or sulfur.

A halogen atom is F, Cl, Br, or I. Each halogen atom forms a single bond to a carbon atom of a hydrocarbyl fragment.

A functional group is one of the moieties depicted below.

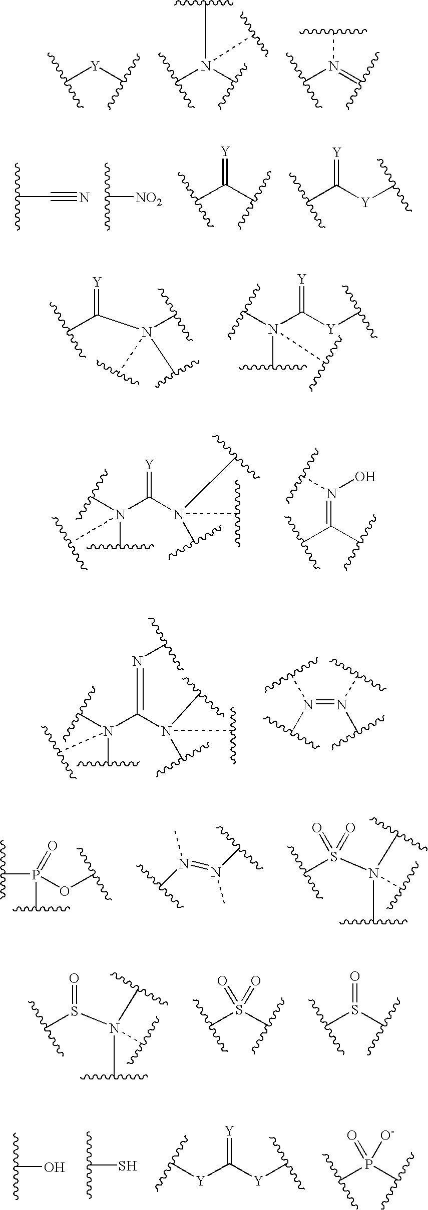

The wavy lines indicate attachment to another atom. Each Y is independently S or O. A functional group bonds directly to a hydrogen or a carbon atom, provided that the following are not present.

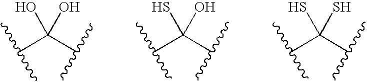

If a functional group is asymmetric, it may be oriented in any way possible. For example, the ester functional group is intended to indicate both of the structures below.

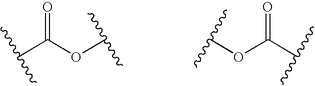

In a substituent, one or more hydrocarbyl fragments and/or one or more functional groups may be incorporated into one or more rings or ring systems.

The dashed lines on the functional groups indicate that any nitrogen atom on a functional group may form an additional bond with another carbon atom, a hydrogen atom, or may form a double bond with one of the depicted bonds so that an ammonium or a quaternary ammonium type of functional group is formed. Thus, the dashed line functional groups actually represent a group of individual functional groups. For example, the functional group:

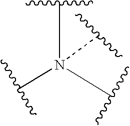

represents the following possible structures:

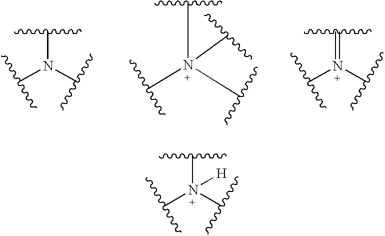

Similarly, the functional group:

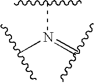

represents the following possible structures:

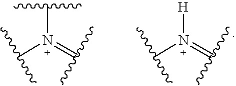

The formula C₀₋₁₂H₀₋₃₀N₀₋₃O₀₋₅P₀₋₂S₀₋₃F₀₋₆Cl₀₋₃Br₀₋₃I₀₋₃ indicates that the structural feature it represents has from 0-12 carbon atoms, from 0-30 hydrogen atoms, from 0-3 nitrogen atoms, from 0-5 oxygen atoms, from 0-2 phosphorous atoms, from 0-3 sulfur atoms, from 0-6 fluorine atoms, from 0-3 chlorine atoms, from 0-3 bromine atoms, and from 0-3 iodine atoms.

In one embodiment, each R¹ is independently H, alkyl, aryl, alkenyl, alkynyl, halo, haloalkyl, hydroxyl, alkoxyl, hydroxyalkyl, alkylcarbonyl, formyl, carboxyl, alkyl carboxylate, alkyl amide, aminocarbonyl, amino, cyano, diazo, nitro, thio, sulfoxyl, sulfonyl, phosphate, or phosphinate.

Z is O or S.

In one embodiment, Z is O.

In another embodiment, Z is S.

X is O, S, or NR²,

In one embodiment, X is O.

In another embodiment, X is S.

In another embodiment, X is NR².

R and R² are independently hydrogen or C₁₋₆ hydrocarbyl.

C₁₋₆ hydrocarbyl is hydrocarbyl having from 1-6 carbon atoms.

C1-6 alkyl is alkyl having from 1-6 carbon atoms.

C1-6 alkenyl is alkenyl having from 1-6 carbon atoms.

C1-6 alkynyl is alkynyl having from 1-6 carbon atoms.

In one embodiment, R is hydrogen, C₁₋₆ alkyl or phenyl.

In another embodiment, R is hydrogen, methyl, ethyl, propyl, or isopropyl.

In one embodiment, R² is hydrogen, C₁₋₆ alkyl or phenyl.

In another embodiment, R² is hydrogen, methyl, ethyl, propyl, or isopropyl.

B has a formula C₀₋₁₂H₁₋₃₀N₀₋₃O₀₋₅P₀₋₂S₀₋₃F₀₋₆Cₗ₀₋₃Br₀₋₃I₀₋₃, and is hydrogen, hydrocarbyl, heterohydrocarbyl, substituted or unsubstituted aryl, substituted or unsubstituted heteroaryl, or a combination thereof.

Thus, B may be hydrocarbyl, as described above.

B may also be heterohydrocarbyl. Heterohydrocarbyl consists of 1) one or more hydrocarbyl fragments, 2) one or more of: a functional group, halo, or a combination thereof, and 3) any necessary hydrogen atoms.

Examples of heterohydrocarbyl include: —R¹⁰-G¹-R¹¹, —R¹⁰-Hl, -G¹-R¹⁰, -G¹-R¹⁰-Hl, G¹-R¹⁰-G², and G¹-R¹⁰-G²-R¹¹, wherein R¹⁰ and R¹¹ are independently hydrocarbyl or hydrogen (provided that hydrogen is attached to only one C, N, O, or S atom), G¹ and G² are independently functional groups, and Hl is halo.

Additional, examples of heterohydrocarbyl are depicted below, wherein R¹⁰, R¹¹, R¹², and R¹³ are independently hydrocarbyl or hydrogen. Other possibilities exist, but are not depicted here.

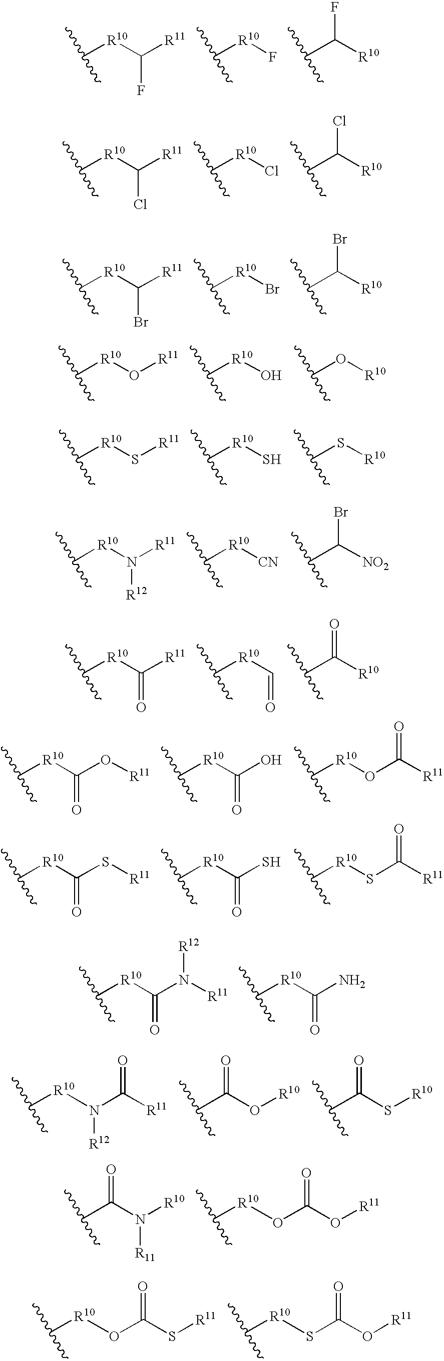

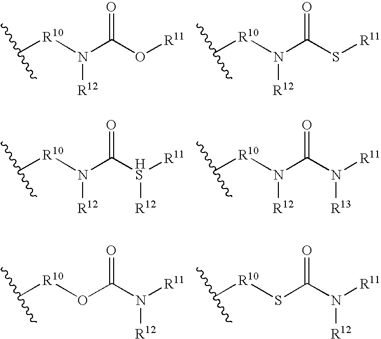

In one embodiment, R¹⁰, R¹¹, R¹² and R¹³ are independently methyl, ethyl, propyl, isopropyl, butyl (any isomer), pentyl (any isomer), hexyl (any isomer), cyclopropyl, cyclobutyl, cyclopentyl, or cyclohexyl.

B may also be substituted or unsubstituted aryl. Aryl has the meaning describe above.

B may also be substituted or unsubstituted heteroaryl. Heteroaryl is an aromatic ring or ring system containing from 1-4 atoms which are part of the ring or ring system (as opposed to being all or part of a substituent) selected from: N, O, S, and combinations thereof. Examples of heteroaryl include pyridine, pyrazine, pyrimidine, pyridazine, triazine, furan, pyrrole, thiophene, imidazole, pyrazole, oxazole, isoxazole, thiazole, isothiazole, oxadiazole, thiadiazole, naphthalene, quinoline, quinoxaline, quinazoline, cinnoline, isoquinoline, benzofuran, indole, benzothiophene, benzimidazole, indazole, benzoxazole, benzisoxazole, benzothiazole, isobenzofuran, isoindole, tetraline, chromane, isochromane, thiochromane, chromene, isochromene, thiochromene, indane, indene, coumarine, coumarinone, and the like.

If the aryl or heteroaryl is substituted, the substituents are the same as those defined above. Examples include: alkyl, aryl, alkenyl, alkynyl, halo, haloalkyl, hydroxyl, alkoxyl, hydroxyalkyl, alkylcarbonyl, formyl, carboxyl, alkyl carboxylate, alkyl amide, aminocarbonyl, amino, cyano, diazo, nitro, thio, sulfoxyl, sulfonyl, phosphate, phosphinate, and the like.

If X is NR², B and R² may together form a ring or ring system. Thus, compounds according to the structural formula below are contemplated.

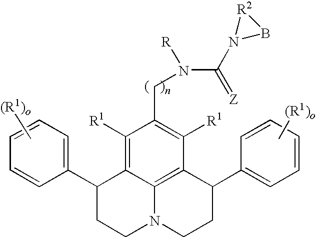

B may also be a combination of one or more of hydrogen, hydrocarbyl, heterohydrocarbyl, substituted or unsubstituted aryl, or substituted or unsubstituted heteroaryl. For example, B may have one of the structures shown below.

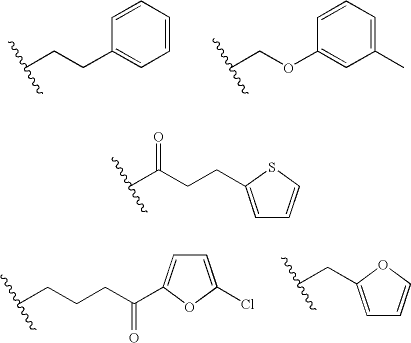

Hypothetical examples of useful compounds are shown below.

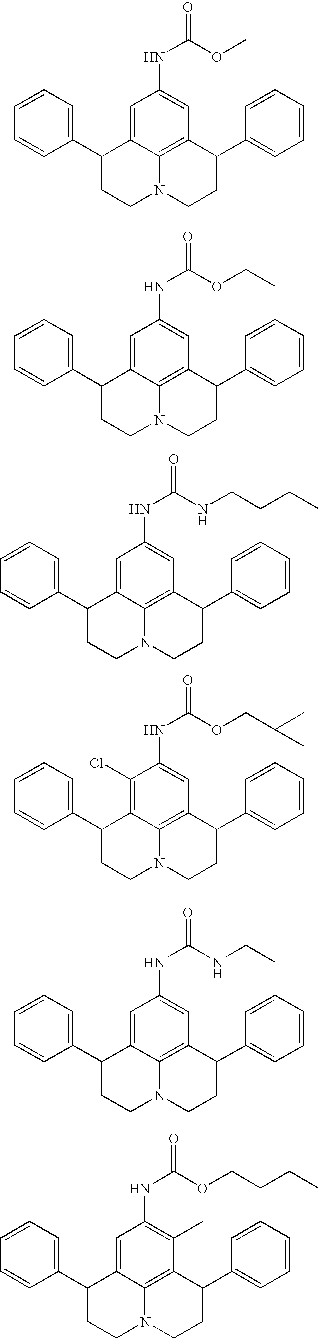

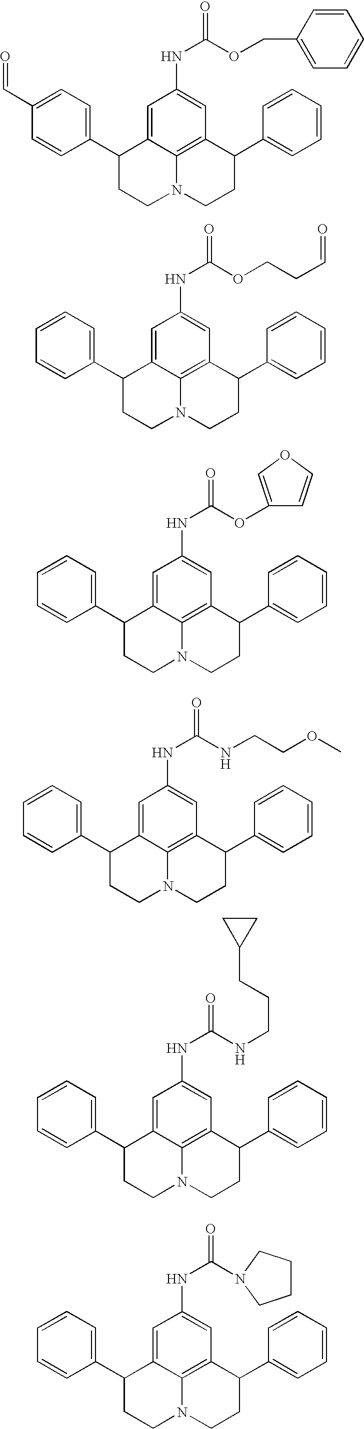

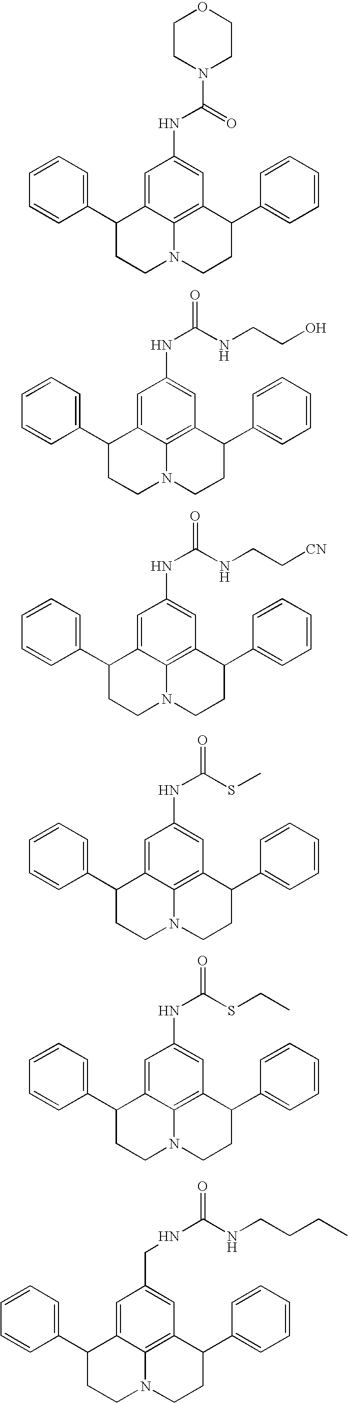

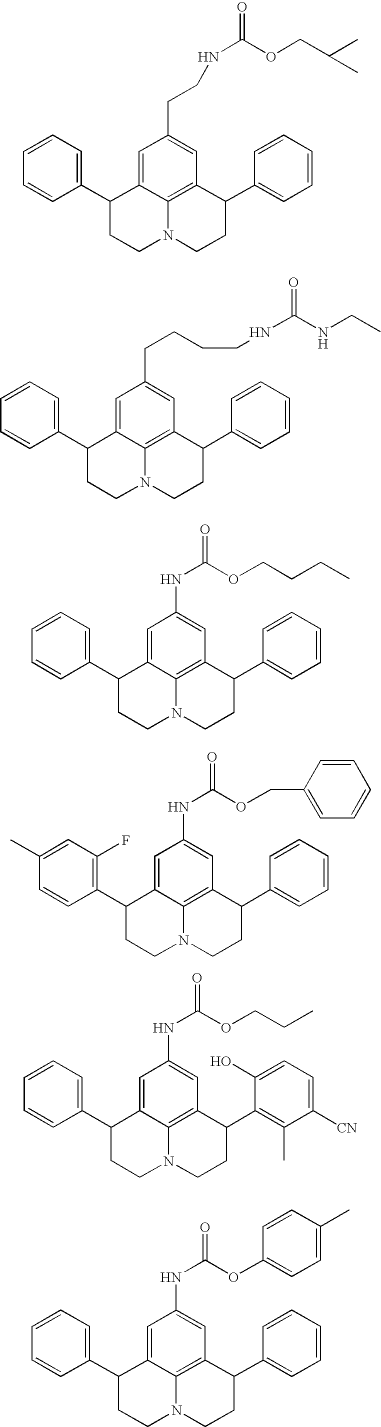

In one embodiment, X is O.

In another embodiment, B is not methyl or ethyl.

In another embodiment, B has at least 3 carbon atoms.

In another embodiment, X is NR².

In another embodiment, R¹ is H, C₁₋₃ alkyl, F, Cl, Br, I, OH, CN, or CF₃.

In another embodiment, n is 0.

In another embodiment, Z is O.

Another embodiment is a compound represented by the structural formula:

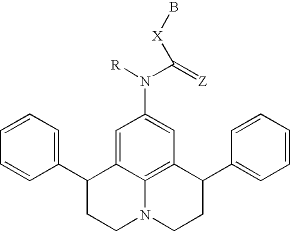

With regard to the structure above:

In another embodiment, Z is O.

In another embodiment, Z is S.

In another embodiment, B is C₁₋₆ alkyl, C₁₋₆ alkenyl, C₁₋₆ alkynyl, C₁₋₆ haloalkyl, phenyl, benzyl, furylmethyl, or wherein X-B is morpholino.

Haloalkyl is alkyl having one or more F, Cl, Br, or I substituents.

C₁₋₆ haloalkyl is C₁₋₆ alkyl having one F, Cl, Br, or I substituents. Examples of haloalkyl include —CH2F, —CH₂CH₂F, —C₃H₆F, —C₄H₈F, —C₅H₁₀F, —C₆H₁₂F, fluorocyclopropyl, fluorocyclobutyl, fluorocyclopentyl, fluorocyclohexyl, —CH₂CH₂Cl, —C₃H₆Cl, —C₄H₈Cl, —C₅H₁₀Cl, —C₆H₁₂Cl, chlorocyclopropyl, chlorocyclobutyl, chlorocyclopentyl, chlorocyclohexyl, —CH₂CH₂Br, —C₃H₆Br, —C₄H₈Br, —C₅H₁₀Br, —C₆H₁₂Br, bromocyclopropyl, bromocyclobutyl, bromocyclopentyl, bromocyclohexyl, —CH₂CH₂I, —C₃H₆I, —C₄H₈I, —C₅H₁₀I, —C₆H₁₂I, iodocyclopropyl, iodocyclobutyl, iodocyclopentyl, iodocyclohexyl,

Morpholino is:

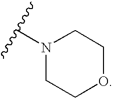

Another embodiment is a compound selected from:

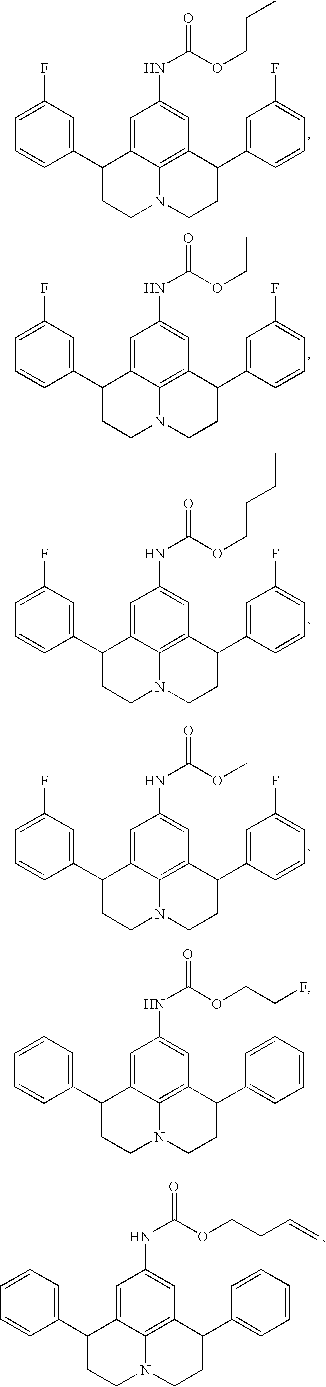

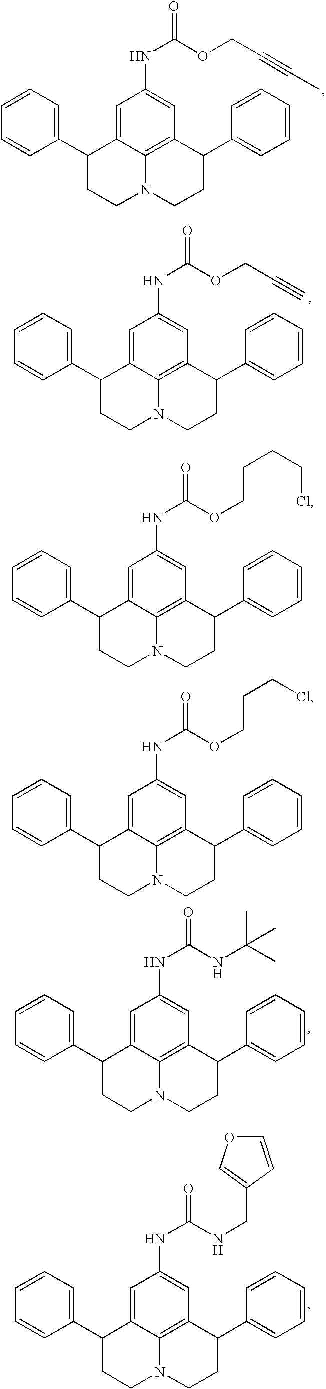

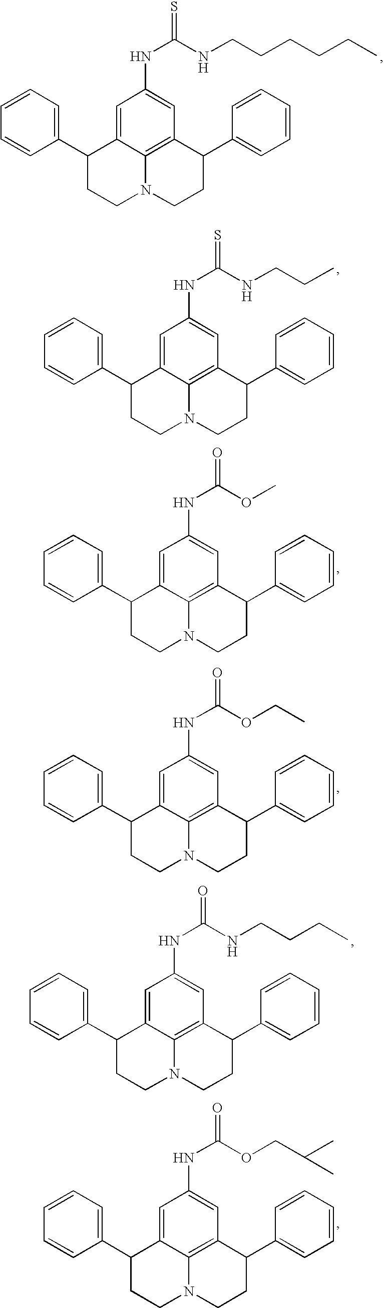

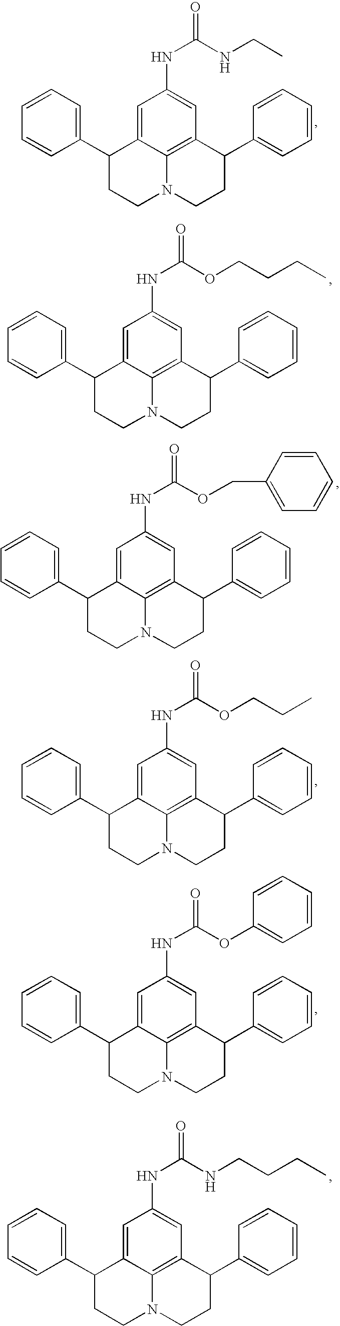

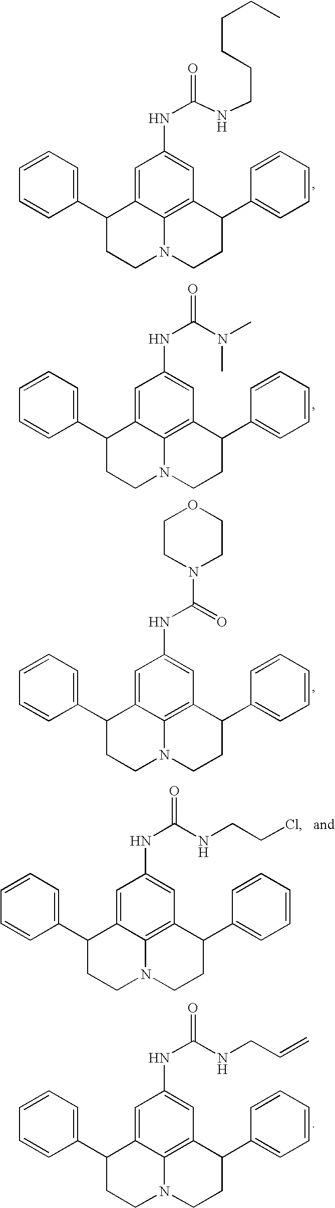

Another embodiment is use of a compound disclosed herein in the manufacture of a medicament for the treatment of a disease or condition in a mammal, said disease or condition selected from glaucoma, dry eye, angiogenesis, cardiovascular conditions and diseases, wounds, and pain.

In another embodiment, the mammal is a human.

Another embodiment is a method of treating a disease or condition comprising administering a compound disclosed herein to a mammal in need thereof, said disease or condition selected from glaucoma, dry eye, angiogenesis, cardiovascular conditions and diseases, wounds, and pain.

In another embodiment, the mammal is a human.

### Synthetic Methods

Reaction Schemes A, B, C and D are examples of useful methods for obtaining the compounds disclosed herein.

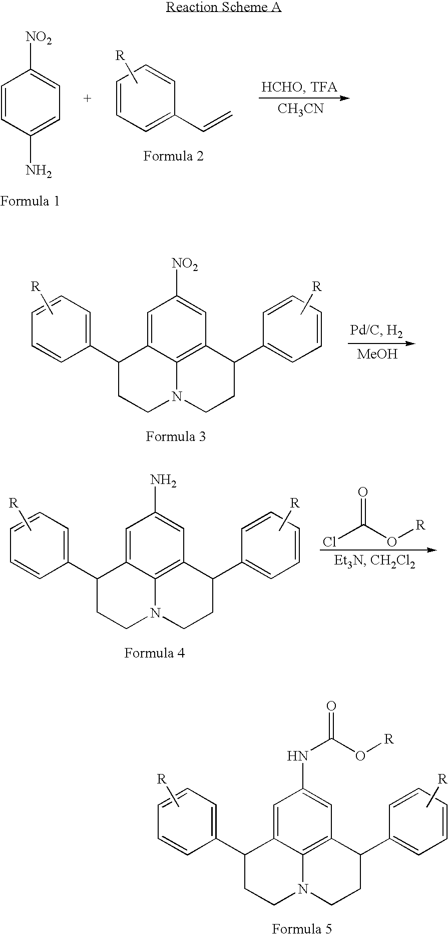

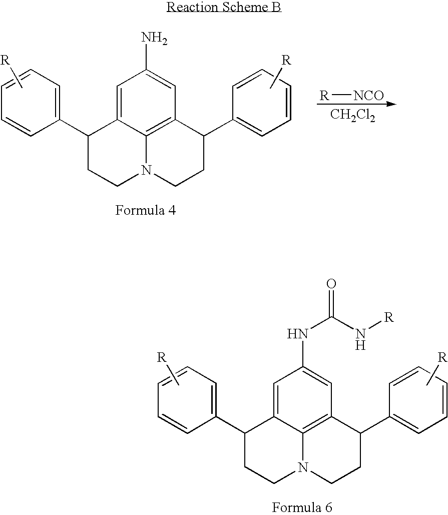

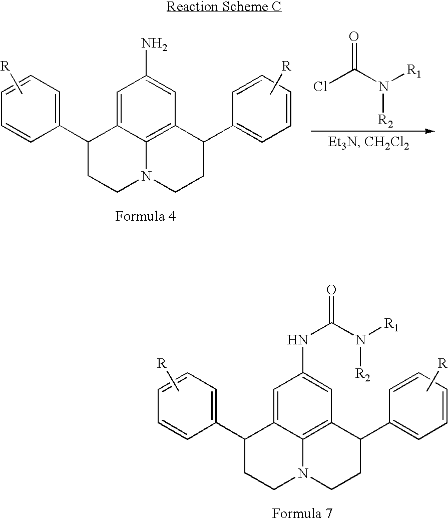

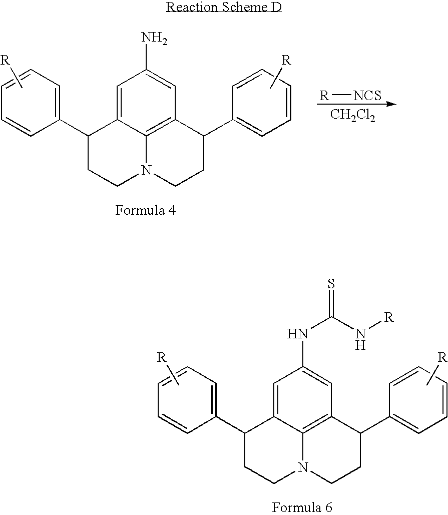

### EXPERIMENTAL EXAMPLES

### Example A

### Method A: Procedure for the methyl 1,7-diphenyl-1,2,3,5,6,7-hexahydropyrido[3,2,1-ij]quinolin-9-ylcarbamate (272)

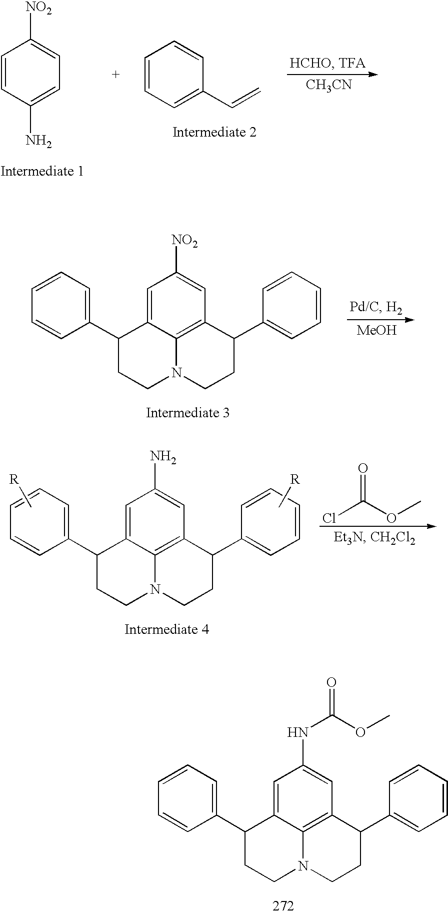

To a solution 4-Nitroaniline (Intermediate 1) (1.8 g, 10 mmol) in acetonitrile (8 mL) was added one equivalent of trifluoroacetic acid (1.14 g, 10 mmol). To this suspension was added with stirring a heterogeneous mixture of styrene (Intermediate 2), (5.74 mL, 50 mmol) and 37% formaldehyde solution (4.06 mL, 50 mmol) under argon, which gave a yellow precipitate. The precipitate failed to redissolve after 30 min. of stirring at room temperature, so the mixture was heated at reflux under argon for further 30 min, during which time the precipitate redissolved. The reaction mixture was cooled to room temperature. The precipitate was filtered and wash with acetonitrile gave yellow solid, 9-nitro-1,7-diphenyl-1,2,3,5,6,7-hexahydropyrido[3,2,1-ij]quinoline, (Intermediate 3), (1.53 g, 41%).

A mixture of 9-nitro-1,7-diphenyl-1,2,3,5,6,7-hexahydropyrido[3,2,1-ij]quinoline, (Intermediate 3), (1.2 g, 7.06 mmol), in MeOH (100 mL) was subjected to hydrogenation reaction by the action of 10% Pd/C (120 mg) under H₂ balloon at room temperature for 12 h. The mixture was filtered through Celite and freed of solvent under reduced pressure to get 1,7-diphenyl-1,2,3,5,6,7-hexahydropyrido[3,2,1-ij]quinolin-9-amine (Intermediate 4) as a solid, (1.08 g, 98%).

To a solution 1,7-diphenyl-1,2,3,5,6,7-hexahydropyrido[3,2,1-ij]quinolin-9-amine (Intermediate 4), (207 mg, 0.608 mmol) in dichloromethane (10 mL) was added three equivalent of triethyl amine (0.252 mL, 1.8 mmol), followed by methyl chloroformate (0.071 mL, 0.91 mmol) under argon at 0° C. The reaction mixture was then stirred at room temperature for overnight. The mixture was quenched with water (30 mL). The residue was isolated in a typical aqueous workup and purified by MPLC (medium pressure liquid chromatography) using silica gel column with 10 to 15% EtOAc:Hexane to give methyl 1,7-diphenyl-1,2,3,5,6,7-hexahydropyrido[3,2,1-ij]quinolin-9-ylcarbamate, (272), (181 mg 75%). ¹H NMR (300 MHz, CDCl₃) δ ppm 2.00-2.18 (m, 2H) 2.22-2.39 (m, 2H) 3.03-3.22 (m, 4H) 3.50-3.64 (m, 3H) 3.54-3.65 (m, 3H) 4.05-4.23 (m, 2H) 6.61 (br. s., 2H) 7.08-7.38 (m, 10H)

### Ethyl 1,7-diphenyl-1,2,3,5,6,7-hexahydropyrido[3,2,1-ij]quinolin-9-ylcarbamate, 273

### (Method: A)

¹H NMR (300 MHz, CDCl₃) □ ppm 1.11 (t, J=7.03 Hz, 3H) 2.02-2.17 (m, 2H) 2.21-2.37 (m, 2H) 3.04-3.17 (m, 4H) 4.04 (q, 2H) 4.09-4.21 (m, 2H) 6.61 (br. s., 2H) 7.08-7.39 (m, 10H)

### Isobutyl 1,7-diphenyl-1,2,3,5,6,7-hexahydropyrido[3,2,1-ij]quinolin-9-ylcarbamate, 274

### (Method: A)

¹H NMR (300 MHz, CDCl₃) □ ppm 0.94 (d, J=6.74 Hz, 6H) 1.84-2.02 (m, 1H) 2.02-2.17 (m, 2H) 2.20-2.37 (m, 2H) 3.31-3.22 (m, 4H) 4.19-4.14 (m, 2H) 6.63 (br. s., 2H) 7.11-7.24 (m, 5H) 7.25-7.37 (m, 5H)

### Propyl 1,7-diphenyl-1,2,3,5,6,7-hexahydropyrido[3,2,1-ij]quinolin-9-ylcarbamate, 275

### (Method: A)

¹H NMR (300 MHz, CDCl₃) □ ppm 0.85 (t, J=6.89 Hz, 3H) 1.46-1.64 (m, 2H) 2.00-2.18 (m, 2H) 2.21-2.37 (m, 2H) 3.03-3.19 (m, 4H) 3.88-4.00 (m, 2H) 4.09-4.23 (m, 2H) 6.61 (br. s., 2H) 7.07-7.37 (m, 10H)

### Butyl 1,7-diphenyl-1,2,3,5,6,7-hexahydropyrido[3,2,1-ij]quinolin-9-ylcarbamate, 276

### (Method: A)

¹H NMR (300 MHz, CDCl₃) □ ppm 0.87 (t, J=7.0 Hz, 3H) 1.30 (br. s., 2H) 1.42-1.56 (m, 2H) 2.02-2.17 (m, 2H) 2.19-2.39 (m, 2H) 3.03-3.17 (m, 4H) 3.91-4.03 (m, 2H) 4.10-4.22 (m, 2H) 6.61 (br. s., 2H) 7.08-7.38 (m, 10H)

### Phenyl 1,7-diphenyl-1,2,3,5,6,7-hexahydropyrido[3,2,1-ij]quinolin-9-ylcarbamate, 277

### (Method: A)

¹H NMR (300 MHz, CDCl₃) □ppm 2.05-2.19 (m, 2H) 2.21-2.40 (m, 2H) 3.04-3.20 (m, 4H), 4.11-4.24 (m, 2H) 6.71 (br. s., 2H) 6.99-7.44 (m, 15H)

### Benzyl 1,7-diphenyl-1,2,3,5,6,7-hexahydropyrido[3,2,1-ij]quinolin-9-ylcarbamate, 278

### (Method: A)

¹H NMR (300 MHz, CDCl₃) □ ppm 1.91-2.09 (m, 2H) 2.10-2.29 (m, 2H) 2.93-3.13 (m, 4H) 4.01-4.15 (m, 2H) 4.92 (s, 2H) 6.56 (br. s., 2H) 7.01-7.32 (m, 15H)

### Propyl 1,7-bis(3-fluorophenyl)-1,2,3,5,6,7-hexahydropyrido[3,2,1-ij]quinolin-9-ylcarbamate, 094

### (Method: A)

1H NMR (300 MHz, CD₃OD) □ ppm 0.89 (t, 7.0 Hz, H) 1.47-1.67 (m, 2H) 1.99-2.14 (m, 2H) 2.19-2.37 (m, 2H) 3.08 (t, J=5.71 Hz, 4H) 3.91 (t, J=6.74 Hz, 2H) 4.17 (t, J=6.01 Hz, 2H) 6.67 (br. s., 2H) 6.81-7.04 (m, 6H) 7.22-7.38 (m, 2H)

### Ethyl 1,7-bis(3-fluorophenyl)-1,2,3,5,6,7-hexahydropyrido[3,2,1-ij]quinolin-9-ylcarbamate, 093

### (Method: A)

1H NMR (300 MHz, CD₃OD) □ ppm 1.15 (t, 7.0 Hz, H) 1.98-2.16 (m, 2H) 2.18-2.37 (m, 2H) 2.96-3.18 (m, 4H) 4.00 (q, J=7.13 Hz, 2H) 4.19 (t, J=5.86 Hz, 2H) 6.68 (br. s., 2H) 6.77-7.01 (m, 6H) 7.20-7.35 (m, 2H)

### Butyl 1,7-bis(3-fluorophenyl)-1,2,3,5,6,7-hexahydropyrido[3,2,1-ij]quinolin-9-ylcarbamate, 202

### (Method: A)

1H NMR (300 MHz, Acetone-d₆) □ ppm 0.86 (t, 7.0 Hz, 6H) 1.20-1.39 (m, 2H) 2.06-2.15 (m, 2H) 2.21-2.39 (m, 2H) 3.02-3.17 (m, 4H) 3.94 (t, J=6.59 Hz, 2H) 4.22 (t, J=5.86 Hz, 2H) 6.83 (br. s., 2H) 6.88-7.09 (m, 6H) 7.28-7.42 (m, 2H)

### Methyl 1,7-bis(3-fluorophenyl)-1,2,3,5,6,7-hexahydropyrido[3,2,1-ij]quinolin-9-ylcarbamate, 779

### (Method: A)

¹H NMR (300 MHz, CD₃OD) □ ppm 2.01-2.15 (m, 2H) 2.21-2.37 (m, 2H) 3.09 (t, J=5.71 Hz, 4H) 3.57 (s, 3H) 4.18 (t, J=6.15 Hz, 2H) 6.67 (s, 2H) 6.81-7.03 (m, 6H) 7.25-7.38 (m, 2H)

### 2-fluoroethyl 1,7-diphenyl-1,2,3,5,6,7-hexahydropyrido[3,2,1-ij]quinolin-9-ylcarbamate, 095

### (Method: A)

1H NMR (300 MHz, CD₃OD) □ppm 1.99-2.16 (m, 2H) 2.19-2.36 (m, 2H) 3.00-3.16 (m, 4H) 4.09-4.27 (m, 4H) 4.29-4.36 (m, 1H) 4.46-4.52 (m, 1H) 6.66 (br. s., 2H) 7.07-7.21 (m, 6H) 7.21-7.33 (m, 4H)

### But-3-enyl 1,7-diphenyl-1,2,3,5,6,7-hexahydropyrido[3,2,1-ij]quinolin-9-ylcarbamate, 354

### (Method: A)

1H NMR (300 MHz, Acetone-d₆) □ppm 2.04-2.15 (m, 2H) 2.18-2.41 (m, 4H) 3.02-3.18 (m, 4H) 3.89-4.01 (m, 2H) 4.90-5.14 (m, 2H) 5.69-5.88 (m, 1H) 6.78 (br. s., 2H) 7.08-7.25 (m, 6H) 7.23-7.38 (m, 4H)

### But-2-ynyl 1,7-diphenyl-1,2,3,5,6,7-hexahydropyrido[3,2,1-ij]quinolin-9-ylcarbamate, 353

### (Method: A)

1H NMR (300 MHz, Acetone-d₆) □ ppm 1.74 (s, 3H) 2.06-2.13 (m, 2H) 2.19-2.34 (m, 2H) 3.02-3.20 (m, 4H) 4.19 (t, J=6.01 Hz, 2H) 4.52 (q, J=2.54 Hz, 2H) 6.78 (s, 2H) 7.12-7.24 (m, 6H) 7.26-7.35 (m, 4H)

### Prop-2-ynyl 1,7-diphenyl-1,2,3,5,6,7-hexahydropyrido[3,2,1-ij]quinolin-9-ylcarbamate, 352

### (Method: A)

1H NMR (300 MHz, Acetone-d₆) □ ppm 2.05-2.15 (m, 2H) 2.20-2.36 (m, 2H) 2.90 (t, J=2.49 Hz, 1H) 3.00-3.20 (m, 4H) 4.19 (t, J=6.01 Hz, 2H) 4.59 (d, J=2.34 Hz, 2H) 6.78 (s, 2H) 7.11-7.24 (m, 6H) 7.25-7.37 (m, 4H)

### 4-chlorobutyl 1,7-diphenyl-1,2,3,5,6,7-hexahydropyrido[3,2,1-ij]quinolin-9-ylcarbamate, 206

### (Method: A)

1H NMR (300 MHz, Acetone-d₆) □ ppm 1.66-1.96 (m, 4H) 2.06-2.11 (m, 2H) 2.22-2.32 (m, 2H) 3.04-3.17 (m, 4H) 3.58 (t, J=6 Hz, 2H) 3.96 (t, J=6.3 Hz, 2H) 4.19 (t, J=6.3 Hz, 2H) 6.77 (s, 2H) 7.16-7.20 (m, 6H) 7.22-7.33 (m, 4H)

### 3-chloropropyl 1,7-diphenyl-1,2,3,5,6,7-hexahydropyrido[3,2,1-ij]quinolin-9-ylcarbamate, 205

### (Method: A)

1H NMR (300 MHz, CD₃OD) □ ppm 1.91-2.14 (m, 4H) 2.20-2.34 (m, 2H) 3.02-3.15 (m, 4H) 4.07 (t, J=6.01 Hz, 2H) 4.11-4.21 (m, 2H) 6.64 (br. s., 2H) 7.09-7.21 (m, 6H) 7.23-7.31 (m, 4H)

### Example B

### Method B: Procedure for the 1-(1,7-diphenyl-1,2,3,5,6,7-hexahydropyrido[3,2,1-ij]quinolin-9-yl)-3-ethylurea (484)

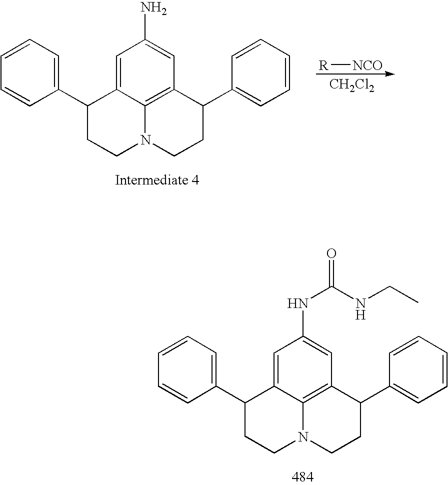

To a solution 1,7-diphenyl-1,2,3,5,6,7-hexahydropyrido[3,2,1-ij]quinolin-9-amine (Intermediate 4), (102 mg, 0.30 mmol) in dichloromethane (15 mL) was added ethyl isocyanate (0.026 mL, 0.33 mmol), mmol) under argon at 0° C. The reaction mixture was then stirred at room temperature for overnight. The solvent was removed under reduced pressure and purified by MPLC (medium pressure liquid chromatography) using silica gel column with 15 to 20% EtOAc:Hexane to get 1-(1,7-diphenyl-1,2,3,5,6,7-hexahydropyrido[3,2,1-ij]quinolin-9-yl)-3-ethylurea (484) (120 mg 97%). ¹H NMR (300 MHz, CDCl₃) □ ppm 0.91 (t, J=7.18 Hz, 3H) 2.05-2.20 (m, 2H) 2.21-2.39 (m, 2H) 2.96-3.28 (m, 6H) 4.07-4.19 (m, 2H) 4.35 (br. s., 1H) 5.58 (s, 1H) 6.43 (s, 2H) 7.06-7.36 (m, 10H)

### 1-Butyl-3-(1,7-diphenyl-1,2,3,5,6,7-hexahydropyrido[3,2,1-ij]quinolin-9-yl)urea, 485

### (Method: B)

¹H NMR (300 MHz, CDCl₃) □ ppm 0.85 (t, J=7 Hz, 3H) 1.04-1.38 (m, 4H) 2.05-2.19 (m, 2H) 2.19-2.38 (m, 2H) 2.89-3.29 (m, 6H) 4.14 (t, J=6.15 Hz, 2H) 4.38 (br. s., 1H) 5.61 (s, 1H) 6.43 (s, 2H) 7.05-7.38 (m, 10H)

### 1-(1,7-diphenyl-1,2,3,5,6,7-hexahydropyrido[3,2,1-ij]quinolin-9-yl)-3-pentylurea, 486

### (Method: B)

¹H NMR (300 MHz, CDCl₃) □ ppm 0.84 (t, J=7.18 Hz, 3H) 1.03-1.33 (m, 6H) 2.04-2.19 (m, 2H) 2.20-2.38 (m, 2H) 2.90-3.28 (m, 6H) 4.07-4.20 (m, 2H) 4.38 (br. s., 1H) 5.58 (s, 1H) 6.43 (s, 5H) 7.07-7.39 (m, 10H)

### 1-(1,7-diphenyl-1,2,3,5,6,7-hexahydropyrido[3,2,1-ij]quinolin-9-yl)-3-hexylurea, 487

### (Method: B)

¹H NMR (300 MHz, CDCl₃) □ ppm 0.86 (t, J=7.1 Hz 3H) 1.06-1.33 (m, 8H) 2.00-2.20 (m, 2H) 2.21-2.40 (m, 2H) 2.93-3.29 (m, 6H) 4.02-4.17 (m, 2H) 4.29-4.45 (m, 1H) 5.57 (br. s., 1H) 6.43 (br. s., 2H) 7.03-7.40 (m, 10H)

### 1-(2-chloroethyl)-3-(1,7-diphenyl-1,2,3,5,6,7-hexahydropyrido[3,2,1-ij]quinolin-9-yl)urea, 769

### (Method: B)

¹H NMR (300 MHz, CD₃OD) □ ppm 1.99-2.15 (m, 2H) 2.19-2.35 (m, 2H) 3.04-3.15 (m, 4H), 3.49 (m, 2H) 3.41-3.50 (m, 2H) 4.11-4.22 (m, 2H) 6.55 (s, 2H) 7.09-7.21 (m, 5H) 7.21-7.32 (m, 5H)

### 1-allyl-3-(1,7-diphenyl-1,2,3,5,6,7-hexahydropyrido[3,2,1-ij]quinolin-9-yl)urea, 773

### (Method: B)

¹H NMR (300 MHz, CD₃OD) □ ppm 2.00-2.14 (m, 2H) 2.20-2.36 (m, 2H) 3.04-3.16 (m, 4H) 3.58-3.69 (m, 2H) 4.11-4.23 (m, 2H) 4.94-5.13 (m, 1H) 5.73 (m, 1H) 6.56 (s, 2H) 7.09-7.21 (m, 5H) 7.23-7.31 (m, 5H)

### 1-tert-butyl-3-(1,7-diphenyl-1,2,3,5,6,7-hexahydropyrido[3,2,1-ij]quinolin-9-yl)urea, 148

### (Method: B)

¹H NMR (300 MHz, CDCl₃) □ ppm 7.12-7.33 (m, 10H) 6.41 (s, 2H) 4.14 (t, J=6 Hz, 2H) 3.14-3.19 (m, 4H) 2.25-2.30 (m, 2H) 2.10-2.17 (m, 2H) 2.25-2.33 (m, 2H)

### 1-(1,7-diphenyl-1,2,3,5,6,7-hexahydropyrido[3,2,1-ij]quinolin-9-yl)-3-(furan-3-ylmethyl)urea, 258

### (Method: B)

¹H NMR (300 MHz, CD₃OD) □ ppm 7.12-7.33 (m, 11H) 6.56 (s, 2H), 6.25-6.27 (m, 1H) 6.08-6.10 (m, 1H) 4.19 (s, 2H) 4.16 (t, J=6 Hz, 2H) 3.07-3.12 (m, 4H) 2.01-2.11 (m, 2H) 2.27-2.29 (m, 2H)

### Example C

### Method C: 3-(1,7-diphenyl-1,2,3,5,6,7-hexahydropyrido[3,2,1-ij]quinolin-9-yl)-1,1-dimethylurea (405)

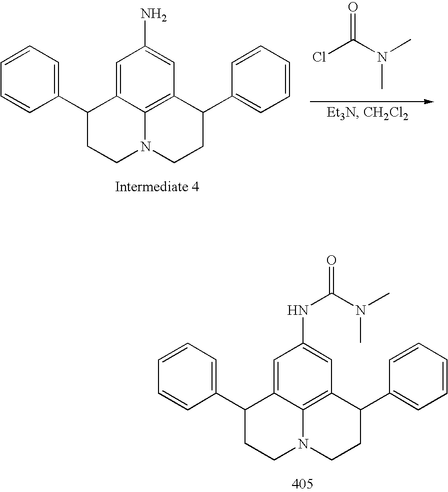

To a solution 1,7-diphenyl-1,2,3,5,6,7-hexahydropyrido[3,2,1-ij]quinolin-9-amine (Intermediate 4), (106 mg, 0.31 mmol) in dichloromethane (10 mL) was added triethyl amine (0.130 mL, 0.933 mmol) followed by dimethylcarbamic chloride (0.043 mL, 0.46 mmol), under argon at 0° C. The reaction mixture was then stirred at room temperature for overnight. The mixture was quenched with water (30 mL). The residue was isolated in a typical aqueous workup and purified by MPLC (medium pressure liquid chromatography) using silica gel column with 10 to 15% EtOAc:Hexane to give 3-(1,7-diphenyl-1,2,3,5,6,7-hexahydropyrido[3,2,1-ij]quinolin-9-yl)-1,1-dimethylurea, (405), (63 mg 49%). ¹H NMR (300 MHz, CDCl₃) □ ppm 2.01-2.16 (m, 2H) 2.20-2.37 (m, 2H) 2.85 (s, 6H) 3.00-3.15 (m, 4H) 4.12-4.25 (m, 2H) 5.78 (s, 1H) 6.62 (s, 2H) 7.10-7.22 (m, 5H) 7.23-7.34 (m, 5H)

### N-(1,7-diphenyl-1,2,3,5,6,7-hexahydropyrido[3,2,1-ij]quinolin-9-yl)morpholine-4-carboxamide, 983

### (Method: C)

¹H NMR (300 MHz, CDCl₃) □ ppm 2.03-2.17 (m, 2H) 2.20-2.38 (m, 2H) 2.92-3.42 (m, 8H) 3.53-3.65 (m, 4H) 3.98-4.20 (m, 2H) 6.52-6.72 (m, 2H) 7.08-7.23 (m, 5H) 7.24-7.37 (m, 5H)

### Example D

### Method D: 1-(1,7-diphenyl-1,2,3,5,6,7-hexahydropyrido[3,2,1-ij]quinolin-9-yl)-3-propylthiourea (255)

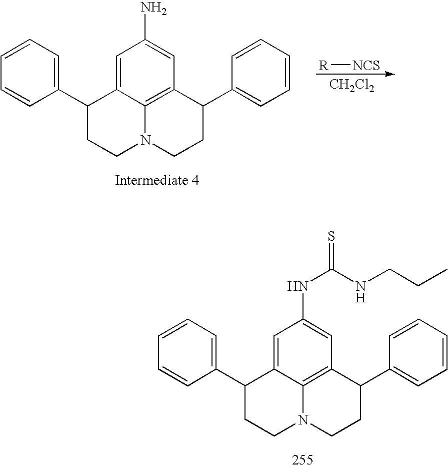

To a solution 1,7-diphenyl-1,2,3,5,6,7-hexahydropyrido[3,2,1-ij]quinolin-9-amine (Intermediate 4), (100 mg, 0.294 mmol) in dichloromethane (15 mL) was added propyl isothiocyanate (0.038 mL, 0.323 mmol) under argon at 0° C. The reaction mixture was then stirred at room temperature for overnight. The solvent was removed under reduced pressure and purified by MPLC (medium pressure liquid chromatography) using silica gel column with 15 to 20% EtOAc:Hexane to get 1-(1,7-diphenyl-1,2,3,5,6,7-hexahydropyrido[3,2,1-ij]quinolin-9-yl)-3-propylthiourea (255) (64 mg 49%). ¹H NMR (300 MHz, CDCl₃) □ppm 7.08-7.34 (m, 10H) 6.39 (s, 2H) 4.11 (t, J=6 Hz, 2H) 3.32-3.46 (m, 2H) 3.16-3.23 (m, 4H) 2.25-2.30 (m, 2H) 2.13-2.17 (m, 2H) 1.32-1.39 (m, 2H, 0.73 (t, J=6 Hz, 3H)

### 1-(1,7-diphenyl-1,2,3,5,6,7-hexahydropyrido[3,2,1-ij]quinolin-9-yl)-3-hexylthiourea, 256

### (Method: D)

¹H NMR (300 MHz, CDCl₃) □ppm 7.07-7.34 (m, 10H) 6.39 (s, 2H) 4.11 (t, J=6 Hz, 2H), 3.46-3.49 (m, 2H) 3.15-3.25 (m, 4H) 2.25-2.32 (m, 2H) 2.11-2.18 (m, 2H) 1.17-1.31 (m, 8H) 0.87 (t, J=6 Hz, 3H)

### In Vitro Data

The in vitro activity of these compounds in the S1P3 receptor was determined using the assay described in paragraph [0067] of United States Patent Application Publication No. 20070232682, which published on Oct. 4, 2007. The results are depicted in the table below.

TABLE 1
Biological Data: Activity potency nM, (IC₅₀), % Inhibition:

| | | | S1P3 |
|---|---|---|---|
| | | | % In- |
| | | S1P3 | hibi- |
| | Structure | IC₅₀ | tion |
| 272 | 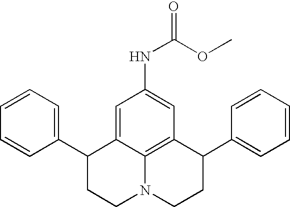 | 8.3 | 102 |
| 273 | 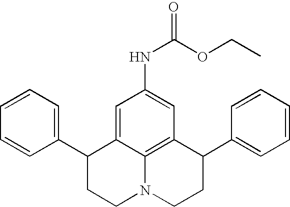 | 63 | 101 |
| 274 | 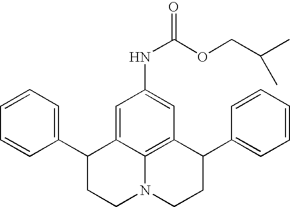 | 64 | 100 |
| 275 | 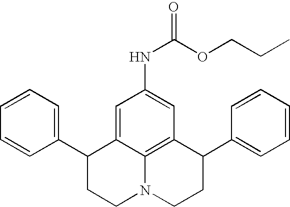 | 12.2 | 100 |
| 276 | 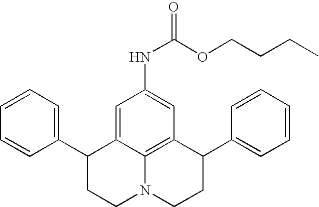 | 8.7 | 101 |
| 277 | 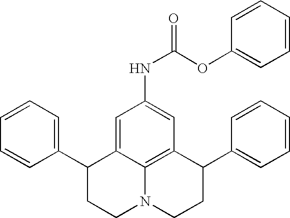 | 901 | 91 |
| 278 | 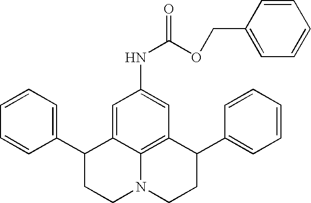 | 66 | 101 |
| 484 | 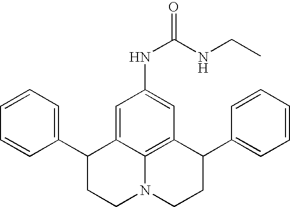 | 77 | 101 |
| 485 | 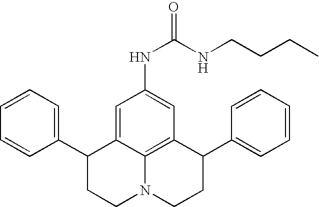 | 52 | 100 |
| 486 | 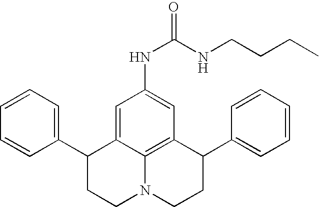 | 82 | 100 |
| 487 | 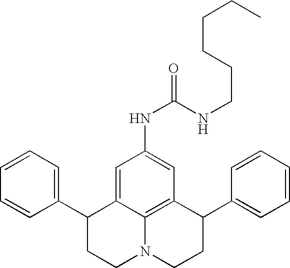 | 301 | 100 |
| 405 | 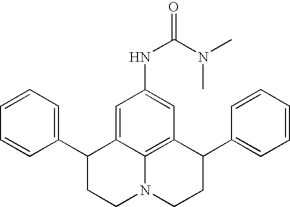 | 131 | 98 |
| 983 | 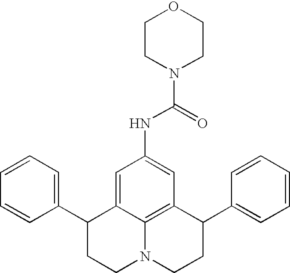 | 130 | 100 |
| 769 | 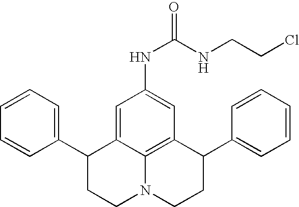 | 374 | 100 |
| 773 | 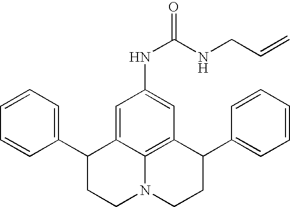 | 160 | 100 |
| 094 | 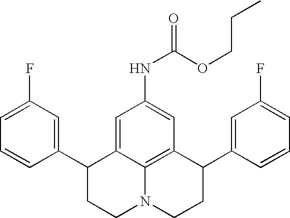 | 234 | 101 |
| 093 | 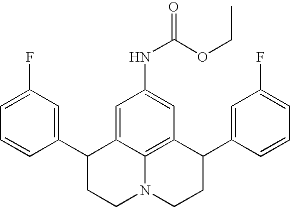 | 48 | 102 |
| 202 | 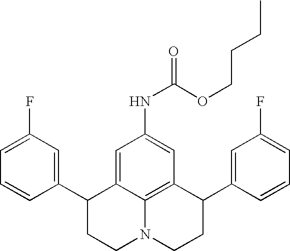 | 272 | 98 |
| 779 | 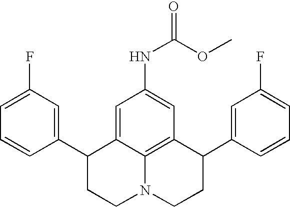 | 23 | 101 |
| 095 | 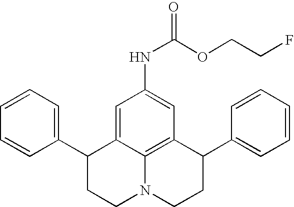 | 56 | 102 |
| 354 | 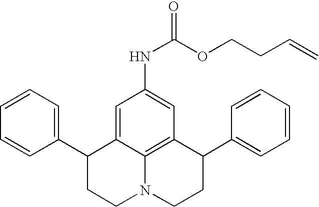 | 16 | 97 |
| 353 | 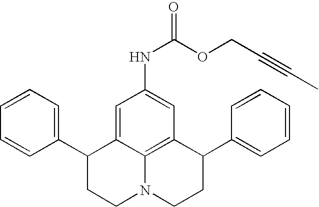 | 13 | 98 |
| 352 | 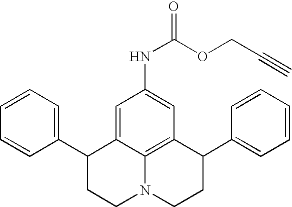 | 40 | 97 |
| 206 | 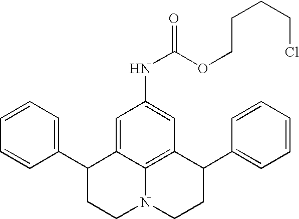 | 62 | 100 |
| 205 | 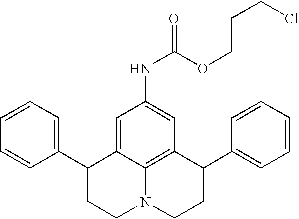 | 8 | 99 |
| 148 | 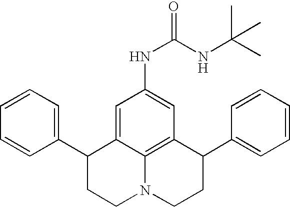 | 38 | 101 |
| 258 | 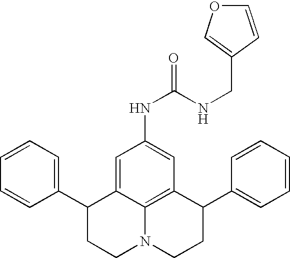 | 59 | 100 |
| 256 | 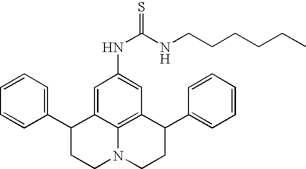 | 78 | 100 |
| 255 | 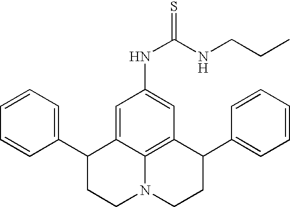 | 153 | 99 |

What is claimed is:

1. A compound represented by the structural formula: 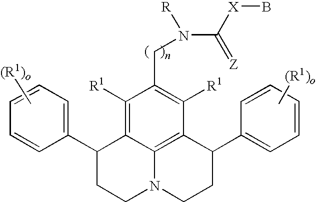
wherein n and o are independently 0, 1, 2, or 3;
R¹ is independently H or a substituent having a formula C₀₋₁₂H₀₋₃₀N₀₋₃₀O₀₋₅P₀₋₂S₀₋₃F₀₋₆Cl₀₋₃Br₀₋₃I₀₋₃;
Z is O or S;
X is O, S, or NR²;
R and R² are independently H or C₁₋₆ hydrocarbyl; and
B has a formula C₀₋₁₂H₁₋₃₀N₀₋₃O₀₋₅P₀₋₂S₀₋₃F₀₋₆Cl₀₋₃Br₀₋₃I₀₋₃, and is H, hydrocarbyl, heterohydrocarbyl, substituted or unsubstituted aryl, substituted or unsubstituted heteroaryl, or a combination thereof, wherein if X is NR², B and R² may together form a ring or ring system.

2. The compound of claim 1 wherein X is O.

3. The compound of claim 2 wherein B is not methyl or ethyl.

4. The compound of claim 2 wherein B has at least 3 carbon atoms.

5. The compound of claim 1 wherein X is NR².

6. The compound of claim 1 wherein R¹ is H, C₁₋₃ alkyl, F, Cl, Br, I, OH, CN, or CF₃.

7. The compound of claim 5 wherein n is 0.

8. The compound of claim 6, further represented by the structural formula:

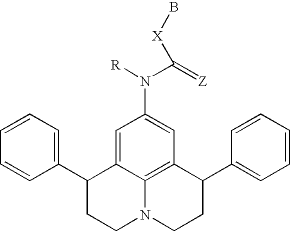

9. The compound of claim 8, wherein Z is O.

10. The compound of claim 9 wherein B is C₁₋₆ alkyl, C₁₋₆ alkenyl, C₁₋₆ alkynyl, C₁₋₆ haloalkyl, phenyl, benzyl, furylmethyl, or wherein X-B is morpholino.

11. The compound of claim 10 selected from:

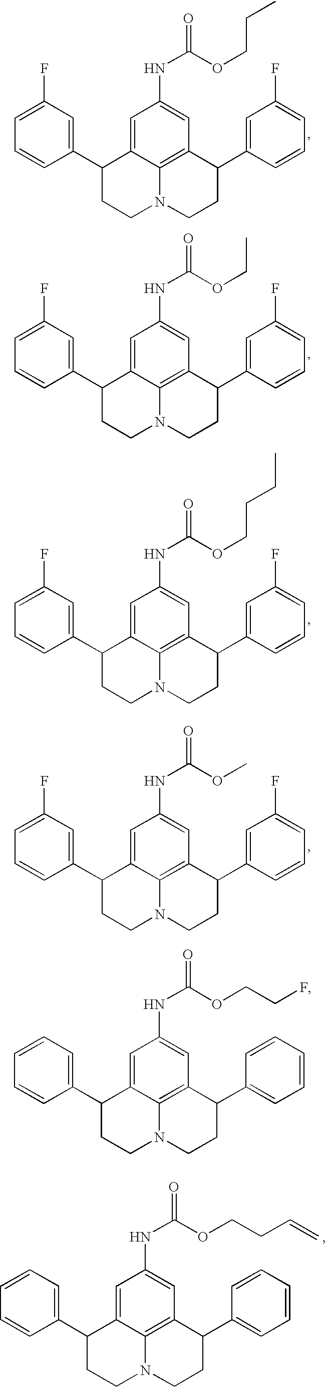

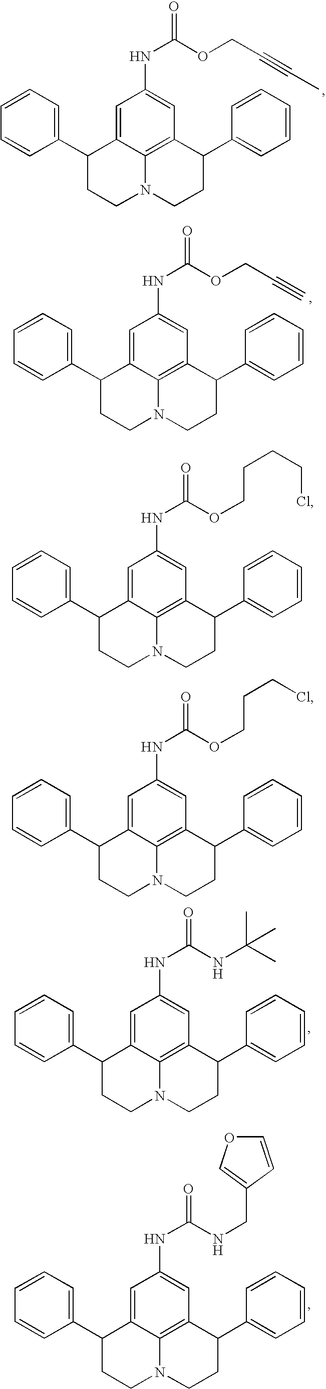

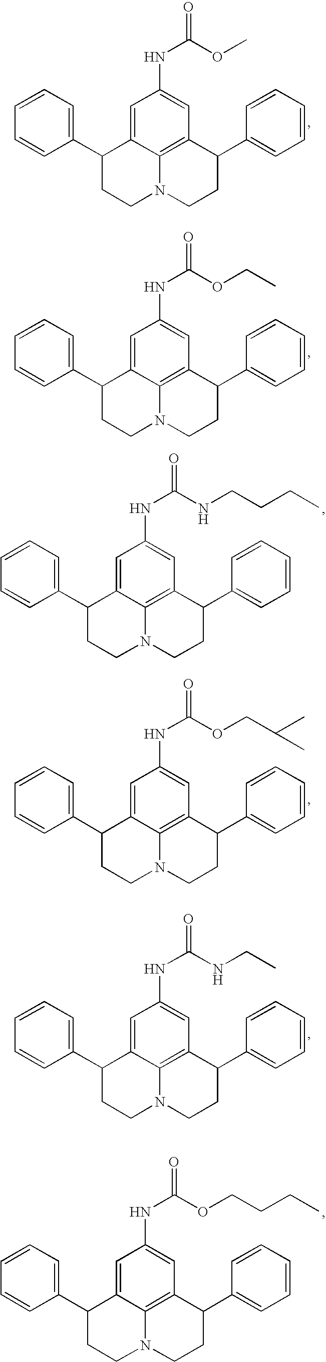

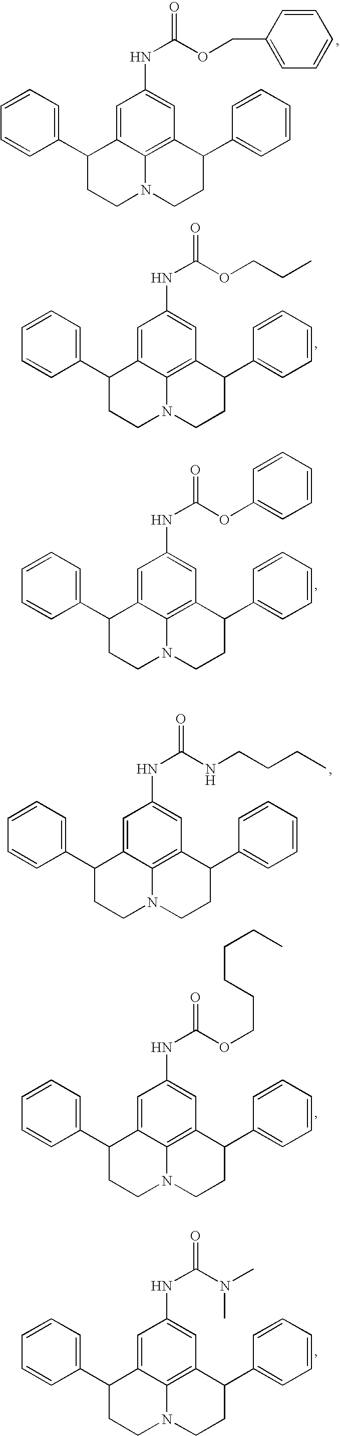

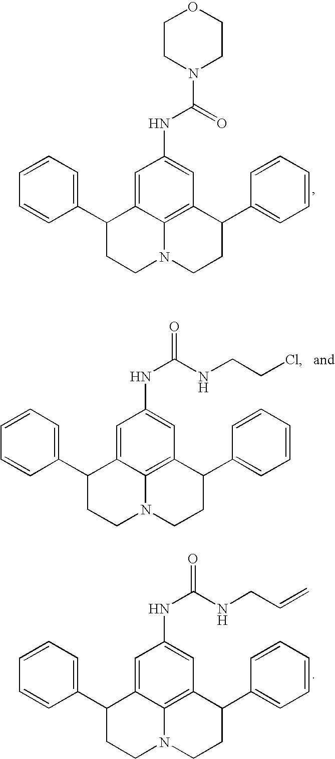

12. The compound of claim 8 wherein Z is S.

13. The compound of claim 12 wherein B is C₁₋₆ alkyl, C₁₋₆ alkenyl, C₁₋₆ alkynyl, C₁₋₆ haloalkyl, phenyl, benzyl, furylmethyl, or wherein X-B is morpholino.

14. The compound of claim 13 selected from:

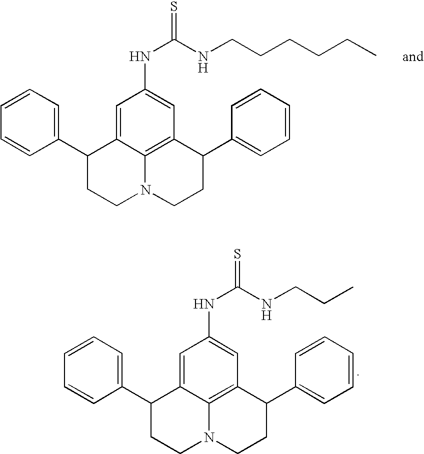

15. A method of treating a disease or condition comprising administering to a subject in need thereof a therapeutically effective amount of a compound of claim 1, said disease or condition selected from glaucoma, dry eye, angiogenesis, cardiovascular conditions and diseases, wounds, and pain.

16. The method of claim 15 wherein the subject is a human.
